(19) United States
(12) Patent Application Publication
(10) Pub. No.: US 2023/0143984 A1
(43) Pub. Date: May 11, 2023

(54) COLLOIDAL CARRIER SYSTEMS FOR TRANSFER OF AGENTS TO A DESIRED SITE OF ACTION

(71) Applicant: Heidelberg University, Heidelberg (DE)

(72) Inventors: Gert Fricker, Dossenheim (DE); Ulrike Müller, Frankfurt (DE); Tobias Köthe, Heidelberg (DE); Vicky Steubler, Dossenheim (DE); Susanne Katharina Klein, Mannheim (DE)

(21) Appl. No.: 17/905,623

(22) PCT Filed: Mar. 22, 2021
(86) PCT No.: PCT/EP2021/057282

§ 371 Date: Sep. 2, 2022

(30) Foreign Application Priority Data
Mar. 20, 2020 (EP) 20164597.5

(51) Int. Cl. A61K 47/69; A61K 38/17; A61K 9/00

(52) U.S. Cl. CPC A61K 47/6911; A61K 38/1716; A61K 47/6933; A61K 47/6915; A61K 9/0085

(57) ABSTRACT

The present invention relates to a drug delivery composition comprising colloidal drug carriers, the composition and a polypeptide for use as a medicament, and in the treatment of neural and neurovascular diseases such as Alzheimer's diseases, and the use of colloidal drug carriers for the production of a drug delivery composition.

### TECHNICAL FIELD

The present invention relates to a drug delivery composition comprising colloidal drug carriers, the composition and a polypeptide for use as a medicament and in the treatment of neural and neurovascular diseases such as Alzheimer's disease, and the use of colloidal drug carriers for the production of a drug delivery composition.

### BACKGROUND OF THE INVENTION

As drug delivery systems, various compounds and systems are discussed and employed depending on the specific selection of target sites. One target site which is subject of intensive research and ongoing discussion in the field is the central nervous system (CNS). Access for drugs to the central nervous system (CNS) is highly restricted due to the presence of the blood-brain barrier (BBB).

Consisting mainly of the capillary endothelial cells connected via tight junctions, it prevents the exchange of most compounds between CNS and blood. Essential nutrients for CNS function are transported by membrane carrier proteins, such as the glucose transporter or amino acid carrier proteins. Thus, homeostasis of the cerebral interstitial fluid is guaranteed.

The exceptional barrier function of the BBB, apart from the tight junctions, is provided by ABC export proteins in the luminal membrane of the capillary endothelial cells, e.g., P-glycoprotein (P-gp, ABCB1), breast cancer resistance protein (BCRP, ABCG2) or the multi-drug resistance protein family (MRPs). Though lipophilic compounds can pass membranes through passive diffusion, the aforementioned transporters recognize most of them as xenobiotica and convey them back into the blood. Many agents, e.g., morphine and phenytoin, are substrates for P-gp which reduces their availability in the CNS drastically.

Targeting of hydrophilic proteins and polypeptides or proteins, including those that have pharmaceutical activity, to the central nervous system faces additional difficulties. For many CNS related diseases, this constitutes a major problem.

Alzheimer's disease as one example of such CNS related diseases is thought to profit from treatment strategies which involve administration and targeting of pharmaceutically active proteins or polypeptides directly to the brain.

It is known that the secreted amyloid precursor protein-alpha (APPsα), being a 612 amino acid protein, has neurotrophic, neuroprotective, neurogenic and synaptogenic properties, stimulates the density of synaptic contacts (dendritic spines) and synaptic plasticity (long-term potentiation=LTP). In addition, it enhances cognitive performance and stimulates both short-term and long-term memory in patients.

A promising new therapeutic approach in Alzheimer's disease may be to increase the brain concentration of APPsα or functional polypeptides derived from it. Animal models have shown that increased intracerebral concentrations of APPsα are able to counteract amyloid-beta induced effects which contribute to the development of clinical symptoms of Alzheimer's disease. Analogous improvements have also been observed in other animal models with reduced synapse density, reduced LTP and decreased memory performance.

However, due to the problematic transfer of polypeptides and proteins to and across the blood-brain barrier, previous approaches were limited to direct injections into the brain or intracranial injections of AAV vectors coding for such polypeptides or proteins. As is apparent, these strategies are associated with the potential for serious complications and significant efforts for the patient as well as for clinical staff.

In view of the aforementioned problems with available drug delivery approaches, it is therefore an object of the present invention to provide a novel and advantageous drug delivery composition for targeted delivery of high loads of protein or polypeptides to their target site. It is further an object of the invention to provide means for drug delivery to the brain which avoids injections or other invasive measures into the brain or cranium, allows systemic administration to obtain targeting to the central nervous system. It is another object of the present invention to provide new products for the treatment of neural and neurovascular diseases such as Alzheimer's disease.

### SUMMARY OF THE INVENTION

The present inventors have dedicated themselves to solving the problem of the present invention and were successful to find novel and useful drug delivery compositions based on colloidal drug carriers for targeted delivery of proteins or polypeptides which overcome the disadvantages and shortcomings of known methods.

The aforementioned objects are solved by the drug delivery compositions as defined by claim 1, being further claimed for use as a medicament as defined by claim 13 and in the treatment of neural and neurovascular diseases as defined by claim 14, by the use of colloidal drug carriers for the production of drug delivery compositions as defined by claim 15, and by a polypeptide for use as a medicament as defined by claim 16 and in the treatment of neural and neurovascular diseases as defined by claim 17. Advantageous developments are the subject matter of the dependent claims.

According to the first aspect of the present invention, a drug delivery composition is provided comprising colloidal drug carriers selected from the group comprising nanoparticles and liposomes, and an agent, wherein the colloidal drug carriers are surface-modified for active targeting to the desired site of action, and wherein the agent is a protein or polypeptide.

According to a preferred embodiment of the first aspect of the present invention, the agent is associated with the colloidal drug carrier, more preferably the agent is encapsulated within the colloidal drug carrier.

According to another preferred embodiment of the first aspect of the present invention, the colloidal drug carriers are nanoparticles.

According to one preferred embodiment of the first aspect of the present invention, the colloidal drug carriers are selected from the group comprising polymersomes or nanospheres.

According to one preferred embodiment of the previous embodiment of the first aspect of the present invention, the nanospheres are formed from poly-butylcyanoacrylate, polylactic acid, poly-glycolic acid or polylactic/glycolic acid.

According to an alternative preferred embodiment of the previous embodiment of the first aspect of the present invention, the polymersomes comprise a copolymer of polyethylene glycol and polycaprolacton (PEG-b-PCL), more preferably the polymersomes are obtained by dual asymmetric centrifugation.

According to another preferred embodiment of the first aspect of the present invention, the colloidal drug carriers are liposomes, more preferably the liposomes comprise cholesterol and distearoyl phosphatidyl choline (DSPC).

According to yet another preferred embodiment of the first aspect of the present invention, the colloidal drug carriers are modified for targeting to cross the blood-brain-barrier, more preferably wherein the colloidal drug carriers are modified with any one of the group comprising ApoE, ApoE fragments, cationized albumin, cell penetrating peptides and/or with antibodies directed against an LRP1-receptor, antibodies directed against a transferrin receptor, antibodies directed against an insulin receptor, or antibodies directed against a Mfsd2a transporter, even more preferably with ApoE or an ApoE fragment, even more preferably with an ApoE4 fragment comprising the sequence of SEQ ID No. 5, particularly preferably with an ApoE4 fragment having the sequence of SEQ ID No. 5.

According to a further preferred embodiment of the first aspect of the present invention, the agent is Amyloid Precursor Protein-α (APPsα) or a polypeptide thereof, more preferably wherein the agent is a polypeptide comprising the C-terminal 16 amino acids of APPsα, even more preferably wherein the agent is a polypeptide comprising the sequence of SEQ ID No. 3 and/or a polypeptide sequence being at least 80% identical to SEQ ID No. 3.

According to a more preferred embodiment, the agent is consisting of the sequence of SEQ ID No. 3 or a polypeptide sequence being at least 80% identical to SEQ ID No. 3, particularly preferably wherein the agent is consisting of the sequence of SEQ ID No. 3.

According to a preferred embodiment of the first aspect of the present invention, the drug delivery composition is suitable for administration to mammals, in particular to humans, more preferably by way of intravenous administration.

According to a second aspect of the present invention, the drug delivery composition according to the first aspect of the present invention is provided for use as a medicament, more preferably wherein the composition is used to release the agent intracerebrally or intracranially.

According to a third aspect of the present invention, the drug delivery composition according to the first aspect of the present invention is provided for use in the treatment of neural diseases or neurovascular diseases, more preferably for use in the treatment of Alzheimer's disease.

According to a preferred embodiment of the third aspect of the present invention, the drug delivery composition is for use in the treatment of Alzheimer's disease, wherein the composition is used for increasing the intracerebral concentrations of Amyloid Precursor Protein-α (APPsα) or a polypeptide thereof.

According to a fourth aspect of the present invention, the use of colloidal drug carriers selected from the group comprising nanoparticles and liposomes is provided for the production of a drug delivery composition comprising agents, more preferably polypeptides or proteins, to the central nervous system.

According to a fifth aspect of the present invention, a polypeptide comprising the sequence of SEQ ID No. 3 and/or a polypeptide sequence being at least 80% identical to SEQ ID No. 3 is provided for use as a medicament, wherein the polypeptide is administered systemically, preferably parenterally, and wherein the polypeptide is targeted to the central nervous system.

According to a sixth aspect of the present invention, a polypeptide comprising the sequence of SEQ ID No. 3 and/or a polypeptide sequence being at least 80% identical to SEQ ID No. 3 is provided for use in the treatment of neural diseases or neurovascular diseases, wherein the polypeptide is administered systemically, preferably parenterally, and wherein the polypeptide is targeted to the central nervous system, preferably for use in the treatment of Alzheimer's disease

### DESCRIPTION OF THE FIGURES

FIG. 1 schematically shows A) a liposome carrier according to one embodiment of the present invention, and B) a schematic representation of the animal experiments.

FIG. 2 depicts the uptake of a liposome carrier according to one embodiment of the present invention into the brain.

FIG. 3 shows cryo-TEM images of inventive liposomes as A) an overview image with the scale bar representing 1 μm, and B) a close-up image with the scale bar representing 100 nm.

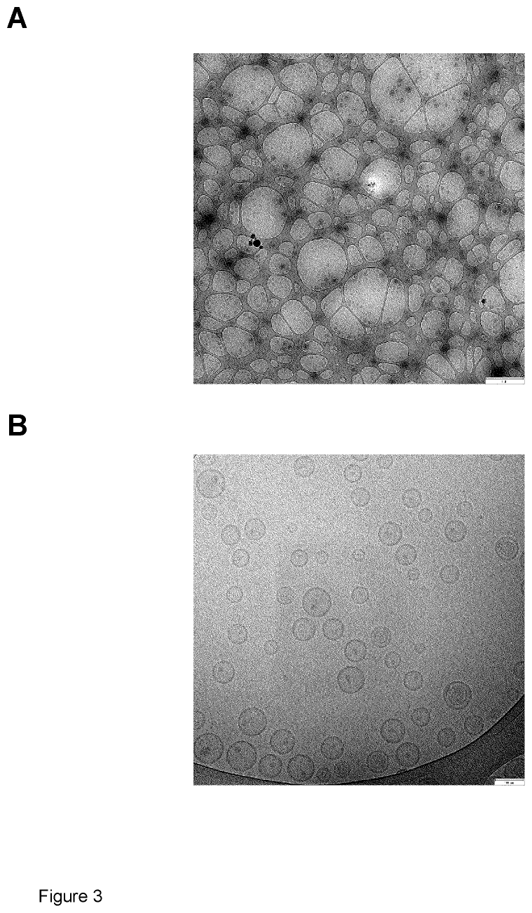

FIG. 4 is a schematic representation of the design of a sandwich ELISA for detection of 2×HA-CTα16 or 2×HA-APPsα (antigen).

FIG. 5 shows (A) encapsulation efficiency (EE) and (B) total load of peptide encapsulated in polymersomes made of PEG-b-PCL (5-b-20 kDa) and PEG-b-PCL (2-b-7.5 kDa) respectively.

FIG. 6 shows (A) AAV-CTα16 constructs enabling CTα16 secretion, wherein a bicistronic construct in which Venus is fused via a T2A site to pre-pro-TRH-CTα16 and an HA-tag is inserted at the N-terminus of CTα16 for easy detection; a vector only encoding the fluorescent protein IckVenus is used as a control vector; TRH: thyrotropin-releasing hormone; (B) ELISA data showing efficient expression of HA-tagged CTα16 in the hippocampus of AAV-CTα16 injected mice, which is not found in animals injected with control vector; (C), (D) spine density of AAV-CTα16 injected mice can be fully restored in basal (C) and midapical (D) dendritic segments.

FIG. 7 shows the peptide sequence of CTα16 (top; SEQ ID NO. 4) that is packed to penetratin-functionalized nanoparticles and intravenously injected into animals, and CTα16-levels peaking two hours after intravenous administration at higher levels than intrahippocampal administration of AAV-CTα16 (bottom).

### DETAILED DESCRIPTION OF THE INVENTION

The present invention is based on the recognition that colloidal carrier systems can be used for targeted delivery of pharmaceutically active agents, such as proteins or polypeptides, to their site of action, in particular the central nervous system. Efficient targeting, which is achieved thereby, can be employed to combat diseases advantageously and in an easier fashion.

According to the present invention, peptide or protein is packaged in nanoparticles consisting of, for example, poly-butylcyanoacrylate or polylactic acid or poly-glycolic acid or polylactic acid/glycolic acid, in polymersomes or in liposomes. All colloidal carriers are surface modified so that active targeting of the blood-brain barrier is achieved (surface modification: e.g. ApoE or ApoE fragments, antibodies (against LRP1 receptor, transferrin receptor, insulin receptor, Mfsd2a transporter) or cationized albumin or cell-penetrating peptides. This is a novel and advantageous way of targeting polypeptides or protein to the central nervous system or other sites in the patient's body. In a specific embodiment of the present invention, the colloidal drug carriers are surface-modified with cell-penetrating peptides (also designated as CPPs), preferably wherein the one or more cell-penetrating peptides are selected from the group consisting of linear or cyclized penetratin (SEQ ID NO: 6; RQIKIWFQNRRMKWKK, derived from Drosophila melanogaster), TAT (transactivator of transcription)-peptide (SEQ ID NO: 7; CGRKKKRRQRRRPPQC, derived from HIV-1), MAP (model amphiphatic peptide) (SEQ ID NO: 8; GALFLGFLGAAGSTMGAWSQPKSKRKV, an artificial peptide), R9 (SEQ ID NO: 9; RRRRRRRRR, an artificial peptide), pVEC (SEQ ID NO: 10; LLIILRRRIRKQAHAHSK-amide, a CPP derived from murine vascular endothelial cadherin), transportan (SEQ ID NO: 11; GWTLNSAGYLLGKINLKALAALAKISIL-amide, derived from the human neuropeptide galanin), and MPG (SEQ ID NO: 12; GALFLGFLGAAGSTMGAWSQPKSKRKV, derived from HIV), combinations thereof, and dimers thereof. In this context, all of the above peptides can be present in a linear or in a cyclized form.

According to one embodiment of the present invention, such CPPs may be attached to a compound being part of the external layer of the colloidal drug carrier. In this context, the term “being part of the external layer of the colloidal drug carrier” is intended to indicate the fact that said compound is integrated into said external layer. In the case of liposomes, the CPP(s) may be attached to a phospholipid integrated into the lipid double layer of the liposome.

Preferably, attachment is covalent attachment. The compound to which the CPPs are attached and which is part of the external layer of the colloidal drug carrier is preferably a suitable lipid or polymer as defined above. Preferably, the CPPs are attached to said compound via a linker. In this context, monomeric CPPs can be covalently attached to a phospholipid or polymer via a linker, or dimerized CPPs, wherein homo- and heterodimers are possible, are covalently attached to a phospholipid or a polymer via a linker.

In the process of making the present invention, the inventors further made use of the recognition that 16 aa C-terminal fragment of APPsα, named CTα16 herein has the same effects in terms of long-term potentiation as the complete protein APPsα (Richter M C et al., 2018 EMBO J, 37, e98335). APPsα with a total sequence length of 612 amino acids had previously been found to have neurotrophic, neuroprotective, neurogenic and synaptogenic properties and stimulates the density of synaptic contacts (dendritic spines) and synaptic plasticity (long-term potentiation=LTP; cf. Fol R et al., 2016 Acta Neuropathol, 131, 247-266; Willer U C et al., 2017 Nat Rev Neurosci 18: 281-298.).

In addition, it enhances cognitive performance and stimulates both short-term and long-term memory. All these beneficial effects have been found to also be caused by the 16 aa fragment. Thus, patients suffering from Alzheimer's disease could significantly profit from proper administration of said fragment to the brain.

The present inventors further found evidence to suggest that CTα16 has therapeutic potential not only against Aβ induced pathology, but also against tau pathology, the other major pathological hallmark of AD. This further supports the plausibility for a high therapeutic potential of the CTα16 peptide (derived from APPsα) for AD and possibly also other neurodegenerative diseases with synaptic deficits.

Administration of said 16 amino acid fragment according to the present invention which targets the active agent to the brain provides even distribution throughout important regions such as cortex and hippocampus. It was observed that the short 16 amino acid fragment causes positive effects similar to the complete APPsα and enhances synaptic plasticity when applied to brain slices in vitro (Richter et al., 2018, supra).

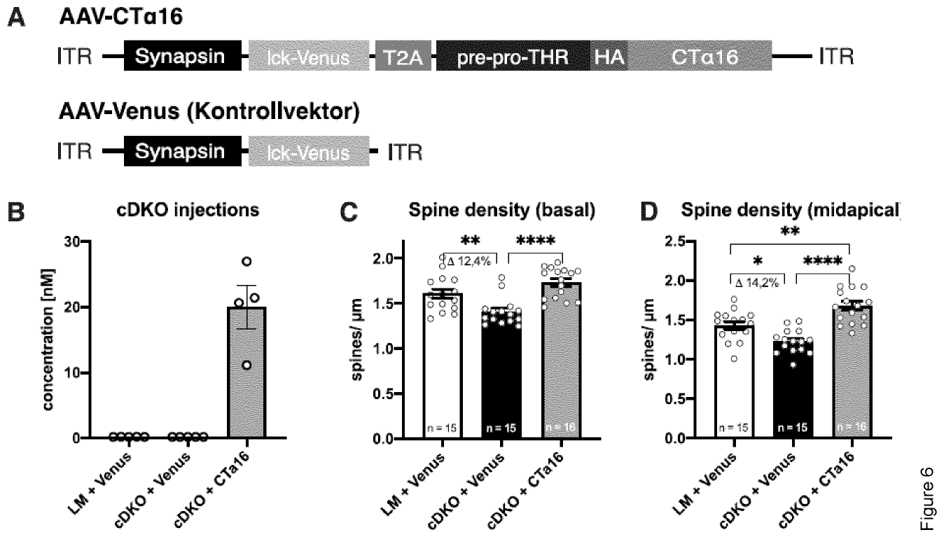

To analyze the concentration of CTα16 in the hippocampus 6 weeks after AAV-CTα16 injections using ELISA, a conditional double knockout line mouse model, termed NexCre cDKO mice (Hick M et al, 2015 Acta Neuropathol, 129, 21-37), which lacks APP and the related APLP2 (APP like protein 2) in excitatory forebrain neurons was used for stereotactic injection of AAV vectors (see FIG. 6A) into the hippocampus of such NexCre cDKO mice.

As can be seen from FIG. 6B, CTα16 concentration obtained in this manner was 20 nM, similar to the range (10 nM) previously used to rescue LTP in vitro. Furthermore, it could be demonstrated that while injections of NexCre cDKO mice with AAV-Venus did not improve spine density, AAV-CTα16 and the concentration obtained therewith fully restored normal spine density in cDKO mice in both basal and apical dendrites of NexCre cDKO mice (cf. FIGS. 6C and 6D).

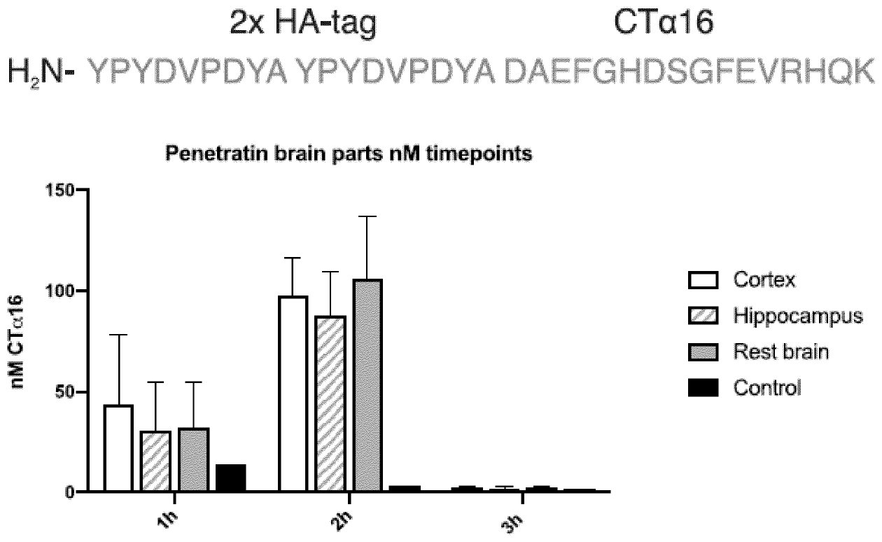

Using nanoparticle injections containing HA-CTα16 peptides according to the present invention, even higher levels of CTα16 ranging from about 30 nM 1 h post injection to about 80-100 nM 2 hrs post injections could be reached (FIG. 7). Thus, the concentration reached by nanoparticle administration exceeds the 20 nM concentration that were shown to lead to pharmacological effects (spine rescue) using AAV-CTα16 delivery.

These experiments show that the CTα16 peptide can not only improve LTP when applied as a recombinant peptide onto brain slices, but that it is also sufficient to restore normal spine density in vivo upon intracranial injection of AAV-CTα16 vectors, expressing CTα16 peptide, into the hippocampus of NexCre-cDKO mice (FIGS. 6 and 7).

The intracranial expression of CTα16 from AAV-CTα16 vectors is considered to be equivalent to an administration by the composition of the present invention, as could be seen by the analysis of CTα16 concentration in the hippocampus as shown in FIGS. 6B and 7. These new findings further demonstrate that CTα16 is sufficient to rescue spine density and corroborates that the small peptide is the major functional domain within APPsα.

Due to the easy administration and the broad and even distribution thereof, it may be expected that patients suffering from neural and neurovascular diseases profit significantly from the novel administration route according to the present invention.

This novel strategy is a promising therapeutic approach in a technical field seeing all clinical studies fail and pharmaceutical companies giving up entire business units relating to this field.

As previously mentioned above, a drug delivery composition is provided by the present invention comprising colloidal drug carriers selected from the group comprising nanoparticles and liposomes, and an agent, wherein the colloidal drug carriers are surface modified for active targeting to the desired site of action, and wherein the agent is a protein or polypeptide.

In the context of the present invention, the agent is preferably associated with the colloidal drug carrier. Association may preferably mean an association between the agent and the external surface of the colloidal drug carrier. Such an association between the agent and the external surface of the colloidal drug carrier may be based on one or more of the following group comprising adsorption, reversible interactions, such as van der Waals, hydrophobic, or lipophilic interaction; a covalent bond; a hydrogen bond; an interaction between ions, an electrostatic interaction, and an aromatic interaction.

More preferably, association of the agent with the colloidal drug carrier means that the agent is encapsulated within the colloidal drug carrier.

According to a preferred embodiment of the present invention, the colloidal drug carriers are selected from the group comprising polymersomes or nanospheres. Preferably, the nanospheres are formed from poly-butylcyanoacrylate, polylactic acid, poly-glycolic acid or polylactic/glycolic acid, more preferably from poly-butylcyanoacrylate.

Nanospheres formed from poly-butylcyanoacrylate may preferably be formed by using miniemulsion polymerization, alternatively preferably by nanoprecipitation.

In an alternative preferred embodiment, nanospheres may be formed according to the disclosure of US patent application US 2017/189345 A1, in particular using the polymer constituents mentioned in paragraphs [0024] to [0027] therein.

Colloidal drug carriers according to the present invention may preferably be surface-modified by surfactants such as polysorbates (in particular polysorbate 80) or poloxamers (in particular P188).

Polymersomes within the present invention preferably comprise one or more of the group comprising diblock copolymers such as polyethylene glycol-b-polycaprolacton (PEG b PCL), polyethylene glycol-b-polylactide (PEG-b-PLA), polyethylene glycol-b-poly(lactic-co-glycolic acid) (PEG-b-PLGA), polyethylene glycol-b-polyglycolid (PEG-b-PGA), poly(dimethylsiloxane)-b-poly(2-methyloxazoline) (PDMS-b-PMOXA), poly(3-caprolactone)-b-poly(2-methacryloyloxyethylphosphorylcholine) (PCL-b-PMPC), polylactid-b-poly(2-methacryloyloxyethylphosphorylcholine) (PLA-b-PMPC), polyethylene glycol-b-polybutadiene (PEG-b-PBD), polyethylene glycol-b-polyethylethylene (PEG-b-PEE), polyethylene glycol-b-polyphenylene sulfide (PEG-b-PPS), polyethylene glycol-b-polytrimethylene carbonate (PEG-b-PTMC) or the like, or triblock copolymers such as poly(lactic-co-glycolic acid)-b-polyethylene glycol-poly(lactic-co-glycolic acid) (PLGA-PEG-PLGA), poly(dimethylsiloxane)-b-poly(2-methyloxazoline)-b-poly(dimethylsiloxane) (PMOXA-b-PDMS-b-PMOXA), polyethylene glycol-b-polypropylene glycol-b-polyethylene glycol (PEG-PPO-PEG) or the like, more preferably the diblock copolymer polyethylene glycol-b-polycaprolacton (PEG-b-PCL).

The average polymer molecular weight fraction of the hydrophilic block portions of the copolymer used for polymersome production is 14 to 45%, more preferably of about 20%. Within the context of the present invention, the average polymer molecular weight fraction of a block portion of the copolymer is the weight percentage relative to the total average polymer molecular weight of the copolymer.

Preferably, the copolymer is in form of a dry powder or a film that may be formed, for example, by dissolving the PEG-b-PCL in methylene chloride and evaporating said solution until the film is formed. Polymersomes may preferably be formed by a method comprising a step of preparing a mixture comprising an aqueous solvent, a copolymer as discussed above and a dispersing aid, following optional steps of homogenizing the mixture and hydrating the copolymer in the mixture, and a subsequent step of processing the mixture prepared in a the previous steps in a dual centrifuge (DC), preferably in a dual asymmetric centrifuge (DAC), to obtain the polymersomes according to the invention.

Furthermore, in the step of preparing a mixture, the dispersing aid may be spherical beads made of glass, metal or a composite material of different materials selected from the above, and volume average particle size diameters (d50) of the beads from 0.1 to 2 mm are preferred. More preferably, the dispersing aid may be spherical ceramic beads with volume average particle size diameters (d50) of 1.0 to 1.2 mm.

Within the context of the present invention, volume average particle size diameter (d50) is preferably analyzed using laser diffraction with Malvern Mastersizer 3000 Particle Size Analyzer as described in ISO Standard 13320 (2009) equipped with a hydro LV sampler and demineralized water as dispersant (Refractive Index=1.33). Material settings: a refractive index of 1.35, an absorption index of 0.60 and a density of 1 g/cm³. Sample is measured 3 times using continuous ultrasonic (setting at 50%) having a measurement loop of 30 sec using red light (630 nm) and 30 sec using blue light (470 nm). Average result will be reported as volume average particle size d50. D50 is defined as the particle size for which 50 percent by volume of the particles has a size lower than the d50.

Other methods for determining particle sizes may be used herein that are commonly known in the art and form part of the common general knowledge as shown in, for example, Kirk-Othmer, Encyclopedia of Chemical Technology, 4th edition, John Wiley & Sons, New York (US), 1997, vol. 22, pages 256 to 278.

For the preparation of the mixture for producing polymersomes, preferably, a composition of the mixture comprising between 0.5 and 40 wt % copolymer, 4.5 and 60 wt % aqueous solution and 20 and 95 wt % dispersing aid, more preferred 3.64 wt % of copolymer, e.g., PEG-b-PCL, 23.64 wt % of aqueous solution, e.g., PBS and 72.73 wt % of dispersing aid, e.g., ceramic beads or another preferred composition of the mixture comprising 6.67 wt % of copolymer 43.33 wt % aqueous solution and 50 wt % of dispersing aid may be used, wherein wt % stands for mass fraction, i.e., percentage of the mass of an individual additive of the mixture relative to the total mass of the mixture.

DCs or DACs are characterized in that a sample, which is conventionally rotated about an rotation axis of a rotor to which the sample is arranged eccentrically in the rotor additionally rotates about its own rotation axis, in contrast to conventional centrifuges in which a sample is only rotated eccentrically about the rotation axis of the rotor in which it is disposed on. Through the second rotation about its own rotation axis, the sample is forced inwards towards the rotation axis of the rotor and thereby thoroughly mixed. DC and DAC differ in that, in a DC, the sample has a similar rotational direction as the rotor in which the sample is disposed on, whereas, in a DAC, a sample has a rotational direction substantially opposite to that of the rotor.

In the step of homogenizing the mixture, the mixture, after being disposed may then preferably subsequently be homogenized by being rotated with a rotational speed in terms of revolutions per minute (rpm). More preferably, the homogenization time during which the mixture is homogenized is at least 1 minute and the rotational speed is between 2000 and 5000 rpm. Particularly preferably, the homogenization time during which the mixture is homogenized is at least 5 minutes and the rotational speed by which the mixture is rotated is about 3540 rpm.

In the optional step of hydrating the copolymer in the mixture, preferably, the mixture is left at room temperature for 10 min or more so that the PEG-b-PCL is hydrated before the step of processing the mixture. More preferably, the time the copolymer is hydrated is at least 30 minutes or the step of hydrating the copolymer in the mixture is omitted, as long as the PEG-b-PCL (or any other copolymer) is properly hydrated.

Preferably, in the step of processing the mixture, similar to the step of homogenizing the mixture described above, the mixture is disposed preferably in a DC, more preferably in a DAC. Consequently, the mixture is processed for at least 10 min by being rotated with a rotational speed of 2000 to 5000. More preferably, the time the mixture is processed is at least 20 minutes, particularly preferably 30 minutes, and the rotational speed by which the mixture is rotated is 3000 to 4000 rpm, particularly preferably about 3540 rpm.

While processing the mixture, the individual copolymers in the mixture, particularly preferably the diblock copolymer PEG-b-PCL, self-assemble as layers (usually monolayers in the case of triblock copolymers and bilayers in the case of diblock copolymers), consequently closing up spherically, thus forming polymersomes.

In this process of assembling the polymersomes, prior to the completion of polymersome formation, the agent is preferably added to the mixture suitable to be enclosed in or bound to the polymersomes. More preferably, the agent may be added to the mixture at any stage of the method described above.

Preferably, the polymersomes as used herein have a Z-Average size of at most 1000 nm, more preferably at most 600 nm, even more preferably at most 400 nm, and a polydispersity index (PDI) of at most 0.5, more preferably at most 0.3. Particularly preferably, in regard to administration of the polymersomes into extracellular or intracellular space of a subject, i.e., systemic administration, the polymersomes have a Z-Average size of at most 200 nm and a PDI of at most 0.2, which is a requirement to be to be able to cross cell membranes and thus to be particularly interesting as a drug delivery system.

The Z-Average is measured by using dynamic light scattering and is a parameter defined by ISO 22412 as the “harmonic intensity averaged particle diameter” i.e. the average hydrodynamic particle size, whereas the polydispersity index (PDI) is a dimensionless number also calculated by using dynamic light scattering that describes the degree of non-uniformity of a size distribution of particles with values smaller than 0.05 indicate a highly monodisperse particle size and values bigger than 0.7 indicate a very broad particle size (Danaei, M.; Dehghankhold, M.; Ataei, S.; Hasanzadeh Davarani, F.; Javanmard, R.; Dokhani, A.; Khorasani, S.; Mozafari, M. R. Impact of Particle Size and Polydispersity Index on the Clinical Applications of Lipidic Nanocarrier Systems. Pharmaceutics 2018, 10, 57).

Further examples for suitable copolymers which may be used in the present invention as disclosed in the prior art may be taken from Discher, D. E. and Eisenberg, A., Science 2002, 297, 967-973, Meng, F. et al., Macromolecules 2003, 36, 3004-3006, Lee, J. S. and Feijen, J., Journal of Controlled Release 2012, 16, 1473-483, Qi, W et al., Nanoscale, 2013, 5, 10908-10915.

In one preferred embodiment of the present invention, the colloidal drug carriers are liposomes. Liposomes may be prepared from phospholipids having different chain lengths and/or degrees of saturation. Preferably, liposomes according to the present invention may comprise one or more phospholipids of the group comprising DLPC, DMPC, DPPC, DSPC, DOPC, DMPE, DPPE, DOPE, DMPA⋅Na, DPPA⋅Na, DOPA⋅Na, DMPG⋅Na, DPPG⋅Na, DOPG⋅Na, DMPS⋅Na, DPPS⋅Na, DOPS⋅Na, DOPE-Glutaryl⋅(Na)₂, Tetramyristoyl Cardiolipin⋅(Na)₂, DSPE-mPEG-2000⋅Na, DSPE-mPEG-5000⋅Na, DSPE-Maleimide PEG-2000⋅Na, DOTAP⋅CI, or the like, for example in accordance with the disclosure of Marsh, D. 2012 Biophys J, 102, 1079-1087.

Also preferably, phospholipids as disclosed in US patent application US 2017/0143629 A1, in particular paragraph [0032] therein, may be employed. Within the present invention, PEGylated lipids as well as tetraetherlipids are also encompassed.

According to one preferred embodiment of the present invention, the liposomes used as colloidal drug carriers comprise cholesterol and distearoyl phosphatidyl choline (DSPC). Liposomes as colloidal drug carriers according to the present invention may preferably be prepared by using one of the methods comprising dual symmetric centrifugation and dual asymmetric centrifugation, more preferably dual asymmetric centrifugation. Methods for liposome preparation may preferably be carried out according to the techniques disclosed in Massing U et al., 2008 J of Contr Release, 125, 16-24 or Massing U et al., 2017 Liposomes, Angel Catala, IntechOpen, DOI: 10.5772/intechopen.68523.).

For the preparation of liposomes, different lipids dissolved in organic solvents are preferably combined and separated by evaporation of the organic solvent to form a lipid film. Preferably, the peptidic or proteinaceous agent is weighed out onto this dry lipid film and then buffer solution is added for rehydration. As in the preparation of polymersomes as discussed above, ceramic beads are preferably added and vesicles enclosing the peptide are formed by the shear forces developing in the centrifuge.

According to one preferred embodiment, the liposomes consist of 38 mol-% cholesterol and 56 mol-% distearoyl phosphatidyl choline (DSPC). In addition, 5 mol-% of a PEGylated distearoyl phosphatidyl ethanolamine (PEG2000-PE) are preferably added, as this prevents recognition of liposomes by the reticuloendothelial system and thus provides for a longer circulation of the liposomes in the blood flow.

For targeting of the colloidal drug carriers of the present invention to the blood-brain barrier, the colloidal drug carriers are preferably modified for targeting to cross the blood-brain-barrier. More preferably, the colloidal drug carriers are modified with any one of the group comprising Apolipoprotein E (ApoE), ApoE fragments, cationized albumin, cell penetrating peptides and/or with antibodies directed against an LRP1-receptor, antibodies directed against a transferrin receptor, antibodies directed against an insulin receptor, or antibodies directed against a Mfsd2a transporter, even more preferably with ApoE or an ApoE fragment.

According to one specific embodiment, the colloidal drug carriers are preferably modified with an ApoE4 fragment comprising the sequence of SEQ ID No. 5, particularly preferably with an ApoE4 fragment having the sequence of SEQ ID No. 5.

According to one embodiment of the present invention, the synthesis of the ApoE lipid (preferably comprising SEQ ID No. 5) was carried out by a so-called click reaction between the maleimide group of the used lipid (preferably DSPE-PEG(2000)-maleimide) and the thiol group of a cysteine, which is part of the ApoE fragment. The by-products of the reaction were separated and the resulting ApoE-lipid conjugate was used to prepare the modified colloidal drug carriers of the present invention. Preferably, 1 mol-% self-synthesized ApoE lipid, more preferably comprising SEQ ID No. 5, is used therein.

According to a preferred embodiment of the present invention, the agent is Amyloid Precursor Protein-α (APPsα) or a polypeptide thereof, more preferably the agent is a polypeptide derived from the C-terminus of APPsα, even more preferably the agent is a polypeptide comprising the 16 C-terminal amino acids of APPsα, even more preferably wherein the agent is a polypeptide comprising the sequence of SEQ ID No. 3 and/or a polypeptide sequence being at least 80% identical to SEQ ID No. 3. Preferably, the Amyloid Precursor Protein-α (APPsα) is of a mammal origin, more preferably from a human or mouse origin, particularly preferably from a human origin.

According to a more preferred embodiment, the agent is consisting of the sequence of SEQ ID No. 3 or a polypeptide sequence being at least 80% identical to SEQ ID No. 3, particularly preferably wherein the agent is consisting of the sequence of SEQ ID No. 3. The sequence represented by SEQ ID No. 3 is of human origin, corresponds to the 16 C-terminal amino acids of APPsα and has a peptide sequence of Asp Ala Glu Phe Arg His Asp Ser Gly Tyr Glu Val His His Gln Lys in 3-letter code and DAEFRHDSGYEVHHQK in 1-letter code. Alternatively preferably, the agent is consisting of the sequence of SEQ ID No. 4 which is the aforementioned peptide of SEQ ID No. 3 with a 2×HA tag.

According to one preferred embodiment, the agent is Amyloid Precursor Protein-α (APPsα) or a polypeptide thereof as described above, wherein the polypeptide is tagged by two hemagglutinin (2×HA) tags at the amino terminus, preferably the agent consists of the sequence of SEQ ID No. 4 (YPYDVPDYAYPYDVPDYADAEFRHDSGYEVHHQK in 1-letter code).

The determination of percent identity between two sequences as used herein is preferably accomplished by using the mathematical algorithm of Karlin and Altschul (Proc. Natl. Acad. Sci. USA (1993) 90: 5873-5877). Such an algorithm is the basis of the BLASTN and BLASTP programs of Altschul et al. (J. Mol. Biol. (1990) 215: 403-410). BLAST polypeptide searches are performed with the BLASTP program. To obtain gapped alignments for comparative purposes, Gapped BLAST is utilized as described by Altschul et al. (Nucleic Acids Res. (1997) 25: 3389-3402). When utilizing BLAST and Gapped BLAST programs, the default parameters of the respective programs are used.

According to a preferred embodiment of the present invention, polypeptide sequences form part of the invention which consist of or comprise a nucleic acid sequence being at least 80% identical to the individualized protein or polypeptide sequences which are disclosed herein, more preferably at least 85% identical, even more preferably at least 90% identical, particularly preferably at least 95% identical. Of course, 100% identical sequences are most preferred herein.

According to one embodiment of the present invention, the polypeptide sequences encompassed by a given identity of 80%, 85%, 90% or 95% may differ in length to the individualized protein or polypeptide sequences disclosed herein, such as being one or more amino acids shorter or longer, as long as 80%, 85%, 90% or 95% of the amino acids of the sequences disclosed herein are still identical.

Regarding the polypeptides disclosed as part of the present invention, the N-terminal and/or C-terminal amino acid may be modified. For example, the N-terminal amino acid of the polypeptides may be alkylated, amidated, or acylated at the N-terminal amino (H₂N—) group, and, for example, the C-terminal amino acid of the peptides may be amidated or esterified at the C-terminal carboxyl (—COOH) group.

For example, the N-terminal amino group may be modified by acylation to include any acyl or fatty acyl group to form an amide, including an acetyl group (i.e., CH₃—C(═O)— or a myristoyl group. In some embodiments, the N-terminal amino group may be modified to include an acyl group having formula —C(O)R, wherein R is a linear or branched alkyl group having from 1 to 15 carbon atoms, or may be modified to include an acyl group having formula —C(O)R¹, wherein R¹ is a linear alkyl group having from 1 to 15 carbon atoms.

The C-terminal amino acid of the peptides may also be chemically modified. For example, the C-terminal carboxyl group of the C-terminal amino acid may be chemically modified to include an amino group in place of the hydroxyl group. (i.e., amidated). In one embodiment, the C-terminus may be amidated by an amine of the formula NH₃, or RNH₂, or R₂NH. Amidated forms of the peptides wherein the C-terminus has the formula CONH₂ are preferred.

Also, the C-terminus of the polypeptides described herein may be in the form of the underivatized carboxyl group, either as the free acid or an acceptable salt, such as the potassium, sodium, calcium, magnesium, or other salt of an inorganic ion or of an organic ion. The carboxyl terminus may also be derivatized by formation of an ester with an alcohol of the formula ROH.

In one embodiment of the present invention, the C-terminus of SEQ ID No. 3 is amidated. In another embodiment of the present invention, the C-terminus of SEQ ID No. 4 is amidated.

The present invention further extends to agents such as antibodies, enzymes, growth factors, and peptides.

In particular, enzymes may preferably be selected from Iduronidase, Arylsulfatases, Heparan sulfate sulfamidase, Acetylglucosamidase, Glucuronidase, and Glucocerebrosidase. Growth factors may preferably be selected from glial-derived neurotrophic factor (GDNF) and brain-derived neurotrophic factor (BDNF). A peptide to be used within the context of the present invention may preferably be the vasoactive intestinal peptide. The agent may further preferably be selected from TNF-receptor (decoy receptor) and TNFα-Inhibitors.

According to one embodiment, the agent is a proteinaceous agent with a molecular weight of at most 15 kDa, preferably at most 10 kDa, more preferably at most 5 kDa, particularly preferably at most 3 kDa. According to another embodiment, the agent is a proteinaceous agent having at most 150 amino acids, preferably at most 100 amino acids, more preferably at most 50 amino acids, particularly preferably at most 20 amino acids.

Other agents which may preferably be used are selected from the group comprising human growth hormone, growth hormone releasing hormone, growth hormone releasing peptide, interferons, colony stimulating factors, interleukins, macrophage activating factor, macrophage peptide, B cell factor, T cell factor, protein A, allergy inhibitor, cell necrosis glycoproteins, immunotoxin, lymphotoxin, tumor necrosis factor, tumor Suppressors, metastasis growth factor, alpha-1 antitrypsin, albumin and fragment polypeptides thereof, apolipoprotein-E, erythropoietin, factor VII, factor VIII, factor IX, plasminogen activating factor, urokinase, streptokinase, protein C, C-reactive protein, renin inhibitor, collagenase inhibitor, Superoxide dismutase, platelet-derived growth factor, epidermal growth factor, osteogenic growth factor, bone stimulating protein, calcitonin, insulin, atriopeptin, cartilage inducing factor, connective tissue activating factor, follicle stimulating hormone, luteinizing hormone, luteinizing hormone releasing hormone, nerve growth factors, parathyroid hormone, relaxin, secretin, Somatomedin, insulin-like growth factor, adrenocortical hormone, glucagon, cholecystokinin, pancreatic polypeptide, gastrin releasing peptide, corticotropin releasing factor, thyroid stimulating hormone, monoclonal or polyclonal antibodies against various viruses, bacteria, or toxins, virus-derived vaccine antigens, octreotide, cyclosporine, rifampycin, lopinavir, ritonavir, Vancomycin, telavancin, oritavancin, dalbavancin, bisphosphonates, itraconazole, danazol, paclitaxel, cyclosporin, naproxen, capsaicin, albuterol Sulfate, terbutaline Sulfate, diphenhydramine hydrochloride, chlorpheniramine maleate, loratidine hydrochloride, fexofenadine hydrochloride, phenylbutaZone, nifedipine, carbamazepine, naproxen, cyclosporin, betamethoSone, danazol, dexamethasone, prednisone, hydrocortisone, 17 beta-estradiol, ketoconazole, mefenamic acid, beclomethasone, alprazolam, midazolam, miconazole, ibuprofen, ketoprofen, prednisolone, methylprednisone, phenytoin, testosterone, flunisolide, diflunisal, budesonide, fluticasone, insulin, acylated insulin, glucagon-like peptide, acylated glucagon-like peptide, exenatide, lixisenatide, dulaglutide, liraglutide, albiglutide, taspoglutide, C-Peptide, erythropoietin, calcitonin, luteinizing hormone, prolactin, adrenocorticotropic hormone, leuprolide, interferon alpha-2b, interferon beta-Ia, Sargramostim, aldesleukin, interferon alpha-2a, interferon alpha-n3alpha-proteinase inhibitor, etidronate, nafarelin, chorionic gonadotropin, prostaglandin E2, epoprostenol, acarbose, metformin, desmopressin, cyclodextrin, antibiotics, antifungal drugs, Steroids, anticancer drugs, analgesics, anti-inflammatory agents, anthelmintics, anti-arrhythmic agents, penicillins, anticoagulants, antidepressants, antidiabetic agents, antiepileptics, antihistamines, antihypertensive agents, antimuscarinic agents, antimycobacterial agents, antineoplastic agents, immunosuppressants, antithyroid agents, antiviral agents, anxiolytic sedatives, hypnotics, neuroleptics, astringents, beta-adrenoceptor blocking agents, blood products and Substitutes, cardiacinotropic agents, contrast media, corticosteroids, cough suppressants, expectorants, mucolytics, diuretics, CNS-active compounds, dopaminergics, antiparkinsonian agents, hemostatics, immunological agents, lipid regulating agents, muscle relaxants, parasympathomimetics, parathyroid calcitonin, prostaglandins, radiopharmaceuticals, sex hormones, steroids, anti-allergic agents, stimulants, anoretics, sympathomimetics, thyroid agents, vasodilators, Xanthines, heparins, therapeutic oligonucleotides, somatostatins and analogues thereof, and pharmacologically acceptable organic and inorganic salts or metal complexes thereof.

In another preferred embodiment, the claimed composition is suitable for administration to mammals, in particular to humans, preferably through systemic administration, more preferably by way of intravenous administration or intranasal administration. According to one preferred embodiment, the claimed composition is suitable for administration by intravenous administration. According to an alternative embodiment, the claimed composition is suitable for administration by intranasal administration. In one preferred embodiment, “suitable for administration” means that the composition is administered by the mentioned route.

The present invention provides the drug delivery composition according to the invention for use as a medicament, preferably wherein the composition is used to release the agent intracerebrally or intracranially. According to one other aspect, the drug delivery composition is provided for use in the treatment of neural diseases or neurovascular diseases, preferably in the treatment of Alzheimer's disease.

Neural diseases or neurovascular diseases according to the present invention may preferably be selected from the group comprising Alzheimer's disease, brain tumors, metastases, glioblastoma, multiple sclerosis, lysosomal storage diseases, stroke, Parkinson's disease, migraine, vasodilatation, ischemic brain damages, traumatic brain damages, neurodegeneration, depression, HIV-associated encephalitis, epilepsy, leucodystrophy, and diseases of the central nervous system. According to a preferred embodiment of the present invention, the neural or neurovascular diseases to be treated with the drug delivery composition of the present invention are those, wherein synaptic repair is still possible.

The present invention also encompasses the use of the drug delivery composition according to the invention as a medicament, and in the treatment of neural diseases or neurovascular diseases, preferably in the treatment of Alzheimer's disease.

In the context of treatment of Alzheimer's disease as referred to in the present invention, the drug delivery composition is preferably used for increasing the intracerebral concentrations of Amyloid Precursor Protein-α (APPsα) or a peptide thereof.

The present invention further provides the use of colloidal drug carriers as defined hereinabove for the production of a drug delivery composition comprising agents, as also further defined hereinabove, which are preferably targeted to the central nervous system.

The present invention further provides a polypeptide comprising the sequence of SEQ ID No. 3 and/or a sequence being at least 80% identical to SEQ ID No. 3 for use as a medicament, wherein the polypeptide is administered systemically, preferably parenterally, and wherein the polypeptide is targeted to the central nervous system.

The present invention further provides a polypeptide comprising the sequence of SEQ ID No. 3 and/or a sequence being at least 80% identical to SEQ ID No. 3 for use in the treatment of neural diseases or neurovascular diseases, preferably for use in the treatment of Alzheimer's disease, wherein the polypeptide is adapted to be targeted to the central nervous system when administered systemically, preferably parenterally.

According to one other aspect of the present invention, use of the polypeptide as a medicament is provided, and use in the treatment of neural diseases or neurovascular diseases, preferably in the treatment of Alzheimer's disease, is provided, wherein the polypeptide is administered systemically, preferably parenterally, and adapted to be targeted to the central nervous system.

All embodiments of the present invention as described herein are deemed to be combinable in any combination, unless the skilled person considers such a combination to not make any technical sense.

### EXAMPLES

### 1) Preparation of Polymersomes

As copolymer, PEG-b-PCL with an average polymer molecular weight of 5-b-20 kDa and a PDI of 1.57 was used in form of dry powder or a film. The film was formed by dissolving the PEG-b-PCL in methylene chloride at 100 mg/mL in a 2 mL reaction tube and evaporated under nitrogen at 50° C. The residual solvent, in particular any organic solvent, was removed under vacuum for at least 1 h. As aqueous solution, PBS and, as dispersing aid, ceramic beads (SiLi Beads Type ZY-E 1.0-1.2 mm, Sigmund-Lindner GmbH, Germany) were used.

For preparing a mixture comprising PEG-b-PCL (5-b-20 kDa), 20 mg of PEG-b-PCL, 130 μL of PBS and 400 mg of ceramic beads were added together.

For preparing another mixture comprising PEG-b-PCL (5-b-20 kDa), 20 mg of PEG-b-PCL, 130 μL of PBS and 150 mg of ceramic beads were added together.

For preparing a mixture comprising PEG-b-PCL (2-b-20 7.5 kDa), 20 mg of PEG-b-PCL, 130 μL of PBS and 150 mg of ceramic beads were added together.

### 1.1) Homogenizing the Mixture and Hydrating the Copolymer in the Mixture

The resulting mixtures were disposed in a DAC and subsequently homogenized for 5 min at a rotational speed of 3540 rpm. After that, the mixtures were left at room temperature for 30 min to hydrate. This approach ensures that the PEG-b-PCL is properly hydrated.

### 1.2) Processing the Mixture in a DAC

After being homogenized and left for hydrating, the mixtures were disposed in the DAC and processed for 30 minutes at a rotational speed of 3540 rpm.

In the mixture comprising, 20 mg of PEG-b-PCL (5-b-20 kDa), 130 μL of PBS and 400 mg of ceramic beads polymersomes were yielded having a Z-Average size of 183±4 nm and a PDI of 0.140±0.003.

In the mixture comprising, 20 mg of PEG-b-PCL (5-b-20 kDa), 130 μL of PBS and 150 mg of ceramic beads polymersomes were yielded having a Z-Average size of 147±4 nm and a PDI of 0.083±0.007.

In the mixture comprising, 20 mg of PEG-b-PCL (2-b-7.5 kDa), 130 μL of PBS and 150 mg of ceramic beads polymersomes were yielded having a Z-Average size of 190±5 nm and a PDI of 0.27±0.01.

### 1.3) Polymersome Encapsulation

For the encapsulating step, polymersomes were prepared using the different mixtures of the method described above.

As the agent to be administered by the polymersomes, the 16 amino acid murine CTα16 peptide (having the sequence of SEQ ID No. 1) with a 2×HA tag having the 34 amino acid sequence according to SEQ ID No. 2 was encapsulated by adding 2 mg of said peptide to the film prior to PBS addition or dissolving it as a 1.8 mg/mL solution in PBS. Due to homology, it can be reasonably assumed that similar effects are observed with the human equivalent.

### 2) Preparation of Nanospheres/Nanoparticles

For the preparation of PBCA nanoparticles via the so-called anionic mini-emulsion polymerisation, a nanoscale emulsion of the oil-in-water type was first produced from two liquids. The oil phase contained 1 ml of the water-insoluble monomer 2-butyl cyanoacrylate (BCA) and 86 μl soybean oil. The water phase of the emulsion consisted of 26 mg sodium lauryl sulphate, 65 mg poloxamer P188 and 5.2 mg of the peptide to be encapsulated (SEQ ID No. 2, 34 aa peptide) dissolved in 5.2 ml 0.1 M phosphoric acid.

The oil phase was added to the water phase and a macroemulsion was formed by repeated pipetting up and down which was stabilized by the surfactants. This macroemulsion was exposed to ultrasound (ultrasonic needle, 70% amplitude) for 4 min under ice cooling. During this process, the emulsion droplets are reduced and unified down to the nanometer range by locally occurring high-energy shock waves due to cavitation.

The contained surfactants stabilize the newly formed droplets, this is commonly referred to as a miniemulsion (Limouzin C et al., 2003 Macromolecules, 36, 667-674). Subsequently, 1.5 ml of the finished miniemulsion at constant stirring (300 rpm) was dripped via a 2 ml syringe with a 24 G cannula into a crimp top glass containing 2.5 ml of an aqueous solution (0.1 M NaOH+0.1 M H₃PO₄) at pH 5.

The pH value of the resulting dispersion was at approximately 3, and the dispersion was subsequently stored overnight at 4° C. to ensure slow and controlled nanoparticle formation. The next day, while stirring constantly (300 rpm), the pH value was raised to neutralization by adding 1.3 ml of 0.1 M sodium hydroxide solution. The neutralized nanoparticle suspension was stored overnight at 4° C. to polymerize residual monomer. The following day, the finished nanoparticle suspension was characterized and used.

### 3) Preparation of Liposomes

The method of dual asymmetrical (DAC) or dual symmetrical (DC) centrifugation was used for this purpose.

The following lipids were first mixed from their stock solutions (9 parts chloroform+1 part methanol) in a reaction vessel by pipetting together:
38 mol-% cholesterol (Sigma Aldrich, Taufkirchen, Germany)
56 mol-% distearyl phosphatidylcholine (DSPC, Lipoid, Ludwigshafen, Germany)
5 mol-% PEGylated distearyl phosphatidylethanolamine (DSPE-PEG2000, Lipoid, Ludwigshafen, Germany)
1 mol-% targeting lipid (=Apolipoprotein E4 peptide fragment, covalently coupled to DSPE-PEG2000 lipid, see below)

The organic solvent was removed from the mixture at 50° C. under continuous nitrogen flow and subsequent drying for at least 30 min under vacuum. During this process, a lipid film was formed on the inner edge of the vessel. The peptide to be encapsulated (2 mg, peptide sequence given above) was added onto this dry lipid film. Buffer solution (DPBS, Gibco) was then added to rehydrate the lipids and dissolve/suspend the peptide. 400 mg ceramic beads (SiLi Beads Type ZY E 1.0 1.2 mm, Sigmund Lindner GmbH, Germany) were added.

Now the first of three centrifugations was performed. When prepared by dual asymmetrical centrifugation, the beads were first centrifuged for 30 min at 3540 rpm, then buffer solution was added again and the mixture was centrifuged for another 5 min at 3540 rpm. Now a buffer solution was added again and the mixture was centrifuged again for 5 min at 3540 rpm. After the third centrifugation, buffer solution was added to a total volume required to achieve a defined lipid concentration. In all experiments, this was 100 mM and the volume required was 190 μl.

The device for dual asymmetric centrifugation was a SpeedMixer™ (DAC 150 FVZ) from Hauschild GmbH & Co KG, Hamm, Germany, which had been modified for longer centrifugation times. When a dual centrifuge was used, the device ZentriMix™ of the company Hettich Zentrifugen, Tuttlingen, Germany was used. The production was carried out in 3 centrifugation steps analogous to the dual asymmetrical centrifugation. However, the centrifugation times were 15 min, 3 min and 3 min. The rotational speed of this unit was 2500 rpm.

During centrifugation, vesicles (liposomes) are formed from the lipids used by the shear forces generated in the process. The amount of enclosed peptide was determined by size exclusion chromatography (Sepharose CL-4B) and HPLC analysis.

### 4) Targeting of Colloidal Carriers to the Blood-Brain Barrier

Targeting of colloidal carriers of the present invention to and over the blood-brain barrier was caused by an ApoE lipid which was synthesized as follows:

The synthesis of the ApoE4 lipid was carried out by a so-called click reaction between the maleimide group of the respective lipid (e.g. DSPE-PEG(2000)-maleimide; Avanti Polar Lipid, Alabaster, Ala., USA) and the thiol group of a cysteine which is part of the ApoE4 fragment of SEQ ID No. 5. For this purpose, lipid and peptide were dissolved in a molar ratio of 1:1.25 in methanol and allowed to react with each other for 48 h with slight shaking (300 rpm) at room temperature. Using a semi-preparative HPLC method, by-products of the reaction could be separated and the thus purified ApoE-lipid conjugate was lyophilized.

For the preparation of carriers adapted to be targeted to the blood-brain barrier, the lyophilisate was dissolved in methanol and mixed as stock solution (5 mM) with the other lipids in the desired ratio (see above).

### 5) Administration to Test Animals

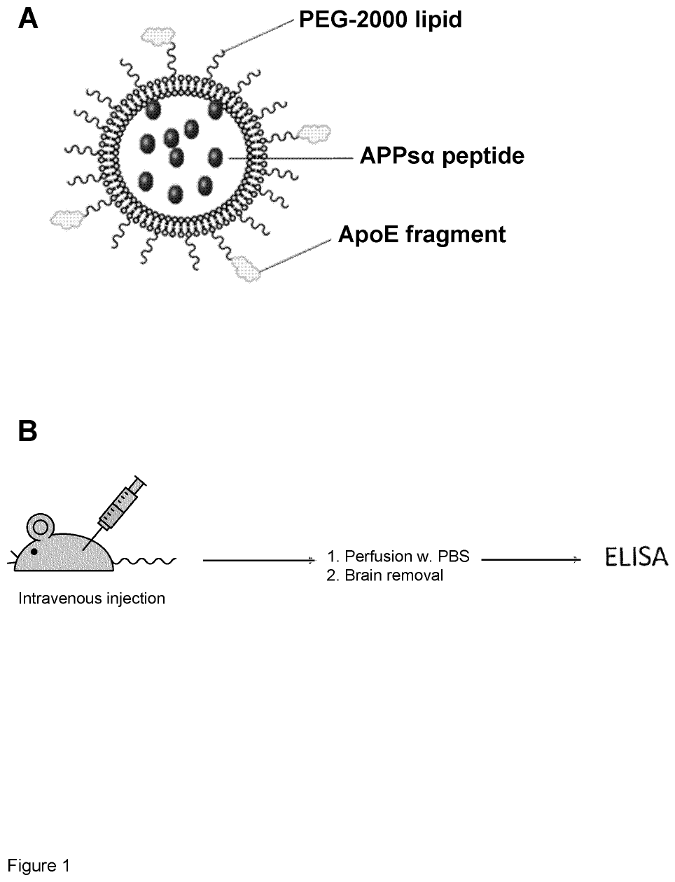

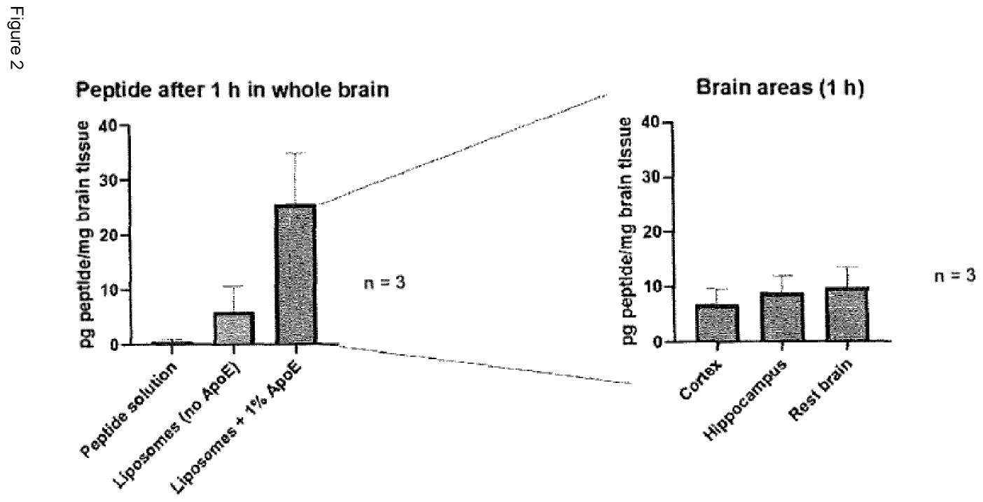

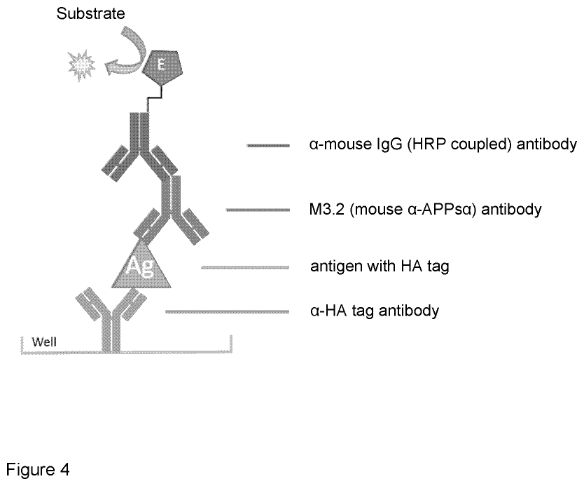

The liposomes produced in this way (see FIG. 1A) were filled as a preparation of 180 μl in insulin syringes (BD Microfine+, U100, 0.3 ml) and 150 μl of this was administered intravenously via the tail vein to so-called Black 6 mice. The brains of the mice were analysed for presence of peptide by ELISA at predetermined times and separately for different regions (Cortex, Cerebellum and Hippocampus; see FIGS. 1B, 2 and 4). The antibodies used included anti-msCTα-16 or anti-msAPPsα antibodies from the supernatant of hybridoma M3.2 (Lab of Prof. Ulrike Willer, Heidelberg), chicken anti-HA tag antibodies (Abcam, Art.Nr ab9111) and HRP goat anti-mouse IgG with low cross reactivity (BioLegend, Art.Nr. 405306).

### B) Devices and Experimental Methods

### Dual Asymmetric Centrifuge (DAC)

The DAC used in the examples is a Speedmixer™ DAC 150 FVZ (Hauschild GmbH & Co KG, Hamm, Germany) with a distance between the rotation axis of the rotor and the rotation axis of the sample of 4.5 cm, a ratio of the rotation of the rotor and the rotation of the sample of approximately 4:1 and a maximum relative centrifugal force or g-force at the rotation axis of the sample of about 600.

### Dynamic Light Scattering (DLS)

Using DLS, the produced Polymersoms were assessed for size and PDI with a Zetasizer Nano ZS (Malvern Instruments Ltd., Worcestershire, United Kingdom) equipped with a 633 nm laser at 173° backscattering. For calculating the mean z-average particle size and PDI, several measurements were taken and were measured using DLS.

### Imaging by Transmission Electron Cryomicroscopy (Cryo-TEM Imaging)

To adequately depict the morphology of nanoparticulate structures of the polymersomes, the polymersomes yielded from the different mixtures were examined using Cryo-TEM Imaging. To do this, a 4 μl aliquot of a sample of polymersomes was adsorbed onto holey carbon-coated grid (Lacey, Tedpella, USA), blotted three seconds with Whatman 1 filter paper and plunge-frozen into liquid ethane at −180° C. using a Vitrobot (FEI company, Hillsboro, USA). Frozen grids were transferred onto a CM FEG microscope (Philips, Amsterdam, Netherlands) using a Gatan 626 cryo-holder (GATAN, Pleasanton, USA). Electron micrographs were recorded at an accelerating voltage of 200 KV using low-dose system (20 to 30 e⁻/Å²) and keeping the sample at −175° C. Defocus values were −4 μm. Micrographs were recorded on 4K×4K TemCam-F CMOS based camera (TVIPS, Gauting, Germany). Nominal magnifications were 50,000× for high magnification images and 5,000× for low magnification images. To determine the dominant particle morphology, particles on low magnification images were counted and classified into monovesicular, solid and “other” depending on their morphology on the micrographs. Polymersomes wall thickness was evaluated by measuring pixel-thickness in GIMP 2.8 (https://www.gimp.org/) and converting to nm using the scale bar pixel-width (data and images not shown).

### Separation by Size Exclusion Chromatography SEC

After being enclosed in polymersomes, substantially any free substance or ingredient was separated from polymersomes using SEC by applying 50 μL of each of the mixtures comprising polymersomes and the substances or ingredients to a gel filtration media in respective columns. The mixture comprising the peptide was applied to the gel filtration media Sepharose CL-4B columns (inner diameter 15 mm, length 90 mm). Consequently, by hydrating and eluting the different columns with PBS, fractions of each column were collected, and fractionation was confirmed and substance or ingredient content was analyzed by using HPLC Analysis for peptide concentrations.

### Determination of Encapsulation—HPLC Analysis

For determining concentrations of peptide in the fractions, an HPLC Agilent HP 0 system (Agilent Technologies, Palo Alto, Calif., USA) with UV detection on a reversed phase column was used. Curve fit was performed using 1/x weighted least squares linear regression (R²>0.99).

### Calculation of Encapsulation Efficiency EE and Load

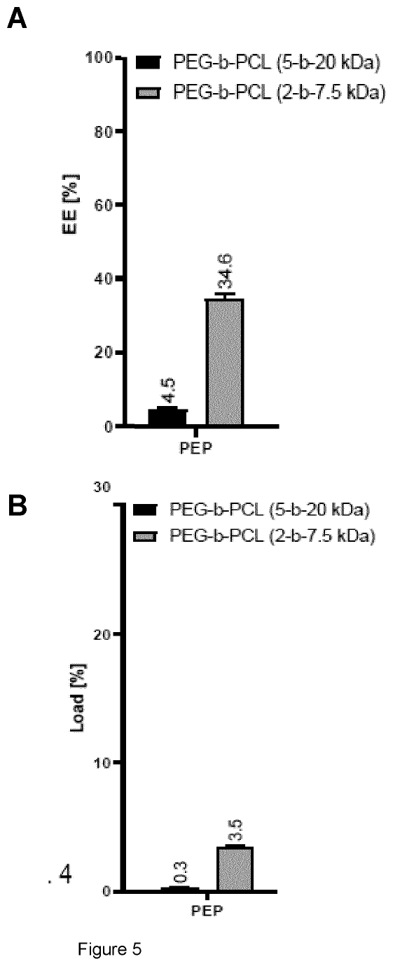

By means of the EE, FIG. 5A shows how much peptide was encapsulated by the polymersomes of the different mixtures. EE was calculated after correcting for all dilutions using the following equation:

EE[%]=100×(concentration of particle fraction)/(concentration of total sample)

The concentration of particle fraction is the concentration of the respective substance in the fraction obtained by SEC and the concentration of total sample the concentration of the substance initially set in the mixture.

FIG. 5B shows the absolute load of the different mixture with peptide, i.e., content of the peptide relative to the mass of the copolymer, which was calculated using the following equation

Load[%]=100×((concentration of particle fraction)×(volume of particle fraction))/(mass of polymer)

The mass of polymer is the mass of the polymer in the fraction.

### C) CTα16 Peptide Administration

Experiments on animals were performed in accordance with the guidelines and regulations set forth by the German Animal Welfare Act and the Regierungspräsidium Karlsruhe, Germany. Generation and genotyping of NexCre cDKO mice (further referred to as cDKO mice) were as described previously (Hick et al, 2015, supra). Genotype of experimental animals: NexCre cDKO (cDKO), APP^{flox/flo}APLP2^{-/-}NexCre^{+/T} and littermate controls (LM controls), APP-WT (=APP^{flox/flox})APLP2^{-/-}.

### AAV Plasmid Design and Vector Production

The mouse APPsα or CTα16 coding sequence (derived from Uniprot: P12023-2) was codon optimized (Geneart, Germany) and then cloned under control of the synapsin promoter into a single-stranded rAAV2-based shuttle vector, as described previously (Fol et al, 2016, supra).

Briefly, the bicistronic DNA constructs harbour a 2A site that connects the cDNA of IckVenus and muAPPsα or CTα16. Venus contains a lymphocyte-specific protein tyrosine kinase (Ick) derived peptide motif which tethers it to the plasma membrane. For easy detection, an N-terminal double HA-tag was inserted downstream of the APP signal peptide (SP) at the N-terminus of APPsα or CTα16.

For CTα16, a pre-pro-TRH site was introduced in front of the HA-tag to ensure proper production of the small CTα16 peptide. The monocistronic control vector, AAV-Venus, encodes only the yellow fluorescent protein Venus. All constructs were packaged into AAV9 capsids. Briefly, viral particles were produced by transient co-transfection of HEK-293 cells with the transfer vector containing the above-mentioned expression cassettes and the helper plasmid pDP9rs.

72 h following transfection, virions were purified and concentrated from cell lysate and supernatant by ultracentrifugation on a iodixanol density gradient followed by buffer exchange to 0.01% pluronic/phosphate-buffered saline (PBS) via a 100 kDa Amicon centrifugal filter unit. Genome copies in the vector stocks were determined by free inverted terminal repeat (ITR)-specific quantitative TaqMan PCR and expressed as genomic copies per μl of concentrated stocks (gc/μl) as described (D'Costa S et al, 2016 Mol Ther Methods Clin Dev; 5: 16019).

### Stereotactic Injection of AAVs

Mice were anesthetized by intraperitoneal injection of sleep mix (Medetomedin: 500 μg/kg, Midazolam: 5 mg/kg, Fentanyl: 50 μg/kg in isotonic NaCl solution) and positioned on a stereotactic frame (World Precision Instruments, USA). Vector particles (either AAV-Venus, AAV-APPsα or AAV-CTα16) were injected into the hippocampus at two injection spots per hemisphere using 1 μl vector stock (titer: 1×10⁹ gc/μl) per spot at a rate of 0.2 μl/min.

When injection was completed, the cannula was left to rest for 1 min to prevent efflux of viral vector solution. Stereotactic coordinates of injection sites from bregma were: anteroposterior (A/P): −2 mm, mediolateral (M/L): ±1 mm, dorsoventral (DN): −2.25 mm and −1.75 mm.

### Spine Density Analysis (Based on Richter et al., 2018; Supra)

### Golgi Staining

Golgi staining was done using the Rapid Golgi Staining Kit (FD NeuroTechnologies, USA) according to the manufacturer's instructions. All procedures were performed under dark conditions. One hemisphere of each mouse was used for Western blot analysis and the other hemsiphere for Golgi staining.

Hemispheres were immersed in 2.5 ml mixtures of equal parts of kit solutions A and B and incubated at room temperature for 2 weeks. After 24 h solution A+B was renewed. Afterwards, brain tissues were stored in solution C at 4° C. for at least 72 h, once exchanged after 24 h. Brains were snap-frozen on dry ice and coronal sections of 100 μm were cut with a cryotome (Hyrax C50, Zeiss, Germany). Each section was mounted with Solution C on an adhesive microscope slide pre-coated with 1% gelatin/0.1% chrome alum on both sides and stained according to the manufacturer's protocol with the exception that RotiClear (Roth, Germany) was used instead of xylene. Finally, slices were cover-slipped with Permount (Thermo Fisher Scientific, USA).

Imaging and Analysis of Spine Density after Golgi Staining

Imaging of second- or third-order dendritic branches of hippocampal pyramidal neurons of area CA1 was done with an Axio Observer Z1 (Zeiss, Germany) for Golgi-stained neurons using a 63×oil objective. Z-stack thickness was hold constant at 130 nm. The number of spines was determined per micrometer of dendritic length (in total 100 μm per neuron) at apical and basal compartments using Neurolucida software (MicroBrightField, USA). Spines in the area around branching points and the soma were excluded from analysis. Five animals per genotype and 3-4 neurons per animal were analyzed blind to genotype and injected viral vector.

### Spine Counts

For evaluation of basal dendritic spine density, at least 3 different randomly chosen dendritic segments of the basal dendritic arbour were imaged. They had to fulfil the following criteria: (1) Lie mostly horizontally to the slice surface, (2) be at least 20 μm away from the soma, (3) have a comparable thickness. The minimum basal dendritic length imaged per neuron was 100 μm.

For evaluation of midapical dendritic spine density, at least 3 different dendritic segments of the apical tree were imaged. Midapical was defined as the middle third of the length of the apical dendrite measured from the origin of the apical dendrite from the soma to the endpoint of the tufts.

Dendritic segments used for evaluation had to fulfil the following criteria: (1) be of second or third order to assure comparable shaft thickness, (2) lie in the middle third of the main apical dendrite (3) be longer than 10 μm. The minimum midapical dendritic length imaged per neuron was 100 μm. Files in the zvi format were imported into ImageJ (NIH) using the BioFormats Importer. After adjusting, images were saved in the TIFF format.

Dendritic spines were manually counted using the Neurolucida and NeuroExplorer software (MicroBrightField, USA) following the criteria of Holtmaat (Holtmaat et al, 2009) with minor modifications: (1) All spines that protruded laterally from the dendritic shaft and exceeded a length of 0.4 μm were counted. (2) Spines that protruded into the z-plane were only counted if they exceeded the dendritic shaft more than 0.4 μm to the lateral side. (3) Spines that bisected were counted as two spines. (4) Spines had to be at least 10 μm away from branching points and the soma. Spine density was expressed as spines per μm of dendrite.

Prior to statistical analysis and blind to genotype, neurons were excluded if the image quality (poor signal to noise ratio) was not sufficient for counting of spines or for deconvolution.

1. Drug delivery composition comprising colloidal drug carriers selected from the group comprising nanoparticles and liposomes, and an agent,
wherein the colloidal drug carriers are surface modified for active targeting to the desired site of action, and
wherein the agent is a protein or polypeptide.

2. Drug delivery composition according to claim 1, wherein the agent is associated with the colloidal drug carrier,
preferably wherein the agent is encapsulated within the colloidal drug carrier.

3. Drug delivery composition according to claim 1, wherein the colloidal drug carriers are nanoparticles.

4. Drug delivery composition according to claim 1, wherein the colloidal drug carriers are selected from the group comprising polymersomes or nanospheres, preferably wherein the nanospheres are formed from poly-butylcyanoacrylate, polylactic acid, poly-glycolic acid or polylactic/glycolic acid.

5. Drug delivery composition according to claim 4, wherein the polymersomes comprise a copolymer of polyethylene glycol and polycaprolacton (PEG-b-PCL),
preferably wherein the polymersomes are obtained by dual asymmetric centrifugation.

6. Drug delivery composition according to claim 1, wherein the colloidal drug carriers are liposomes,
preferably wherein the liposomes comprise cholesterol and distearoyl phosphatidyl choline (DSPC).

7. Drug delivery composition according to claim 1, wherein the colloidal drug carriers are modified for targeting to cross the blood brain barrier,
preferably wherein the colloidal drug carriers are modified with any one of the group comprising ApoE, ApoE fragments, cationized albumin, cell penetrating peptides and/or with antibodies directed against an LRP1-receptor, antibodies directed against a transferrin receptor, antibodies directed against an insulin receptor, or antibodies directed against a Mfsd2a transporter,
more preferably with ApoE or an ApoE fragment, even more preferably with an ApoE4 fragment comprising the sequence of SEQ ID No. 5, particularly preferably with an ApoE4 fragment having the sequence of SEQ ID No. 5.

8. Drug delivery composition according to claim 1, wherein the colloidal drug carriers are surface-modified with cell-penetrating peptides,
preferably wherein the cell-penetrating peptides are selected from the group consisting of linear or cyclized penetratin (SEQ ID NO: 6; RQIKIWFQNRRMKWKK).

9. Drug delivery composition according to claim 1, wherein the agent is Amyloid Precursor Protein-α (APPsα) or a polypeptide thereof,
preferably wherein the agent is a polypeptide comprising the C-terminal 16 amino acids of APPsα, even more preferably the agent is a polypeptide comprising the sequence of SEQ ID No. 3 and/or a polypeptide sequence being at least 80% identical to SEQ ID No. 3.

10. Drug delivery composition according to claim 9, wherein the agent is consisting of the sequence of SEQ ID No. 3 or a polypeptide sequence being at least 80% identical to SEQ ID No. 3, preferably wherein the agent is consisting of the sequence of SEQ ID No. 3.

11. Drug delivery composition according to claim 1, wherein the drug delivery composition is suitable for administration to mammals, in particular to humans,
preferably by way of intravenous administration.

12. Drug delivery composition according to claim 1, wherein the colloidal drug carrier is a liposome which is surface-modified with penetratin (SEQ ID NO: 6; RQIKIWFQNRRMKWKK) and the agent is a polypeptide comprising the sequence of SEQ ID No. 3 and/or a polypeptide sequence being at least 80% identical to SEQ ID No. 3.

13. Drug delivery composition according to claim 1, for use as a medicament,
preferably wherein the composition is used to release the agent intracerebrally or intracranially.

14. Drug delivery composition according to claim 1, for use in the treatment of neural diseases or neurovascular diseases, preferably for use in the treatment of Alzheimer's disease, wherein the composition is more preferably used for increasing the intracerebral concentrations of Amyloid Precursor Protein-α (APPsα) or a peptide thereof.

15. Use of colloidal drug carriers selected from the group comprising nanoparticles and liposomes for the production of a drug delivery composition comprising agents, preferably peptides or proteins, which are more preferably targeted to the central nervous system.

16. Polypeptide comprising the sequence of SEQ ID No. 3 and/or a sequence being at least 80% identical to SEQ ID No. 3 for use as a medicament, wherein the polypeptide is administered systemically, preferably parenterally, and wherein the polypeptide is adapted to be targeted to the central nervous system.

17. Polypeptide comprising the sequence of SEQ ID No. 3 and/or a sequence being at least 80% identical to SEQ ID No. 3 for use in the treatment of neural diseases or neurovascular diseases,
wherein the polypeptide is administered systemically, preferably parenterally, and wherein the polypeptide is adapted to be targeted to the central nervous system,
preferably for use in the treatment of Alzheimer's disease.


SEQUENCE LISTING (12 sequences)

SEQ ID NO: 1 - protein, 16 aa, Mus musculus
  mouse APPs alpha fragment
DAEFGHDSGFEVRHQK

SEQ ID NO: 2 - protein, 34 aa, Artificial Sequence
  mouse APPs alpha fragment with 2xHA tag
YPYDVPDYAYPYDVPDYADAEFGHDSGFEVRHQK

SEQ ID NO: 3 - protein, 16 aa, Homo sapiens
  human APPs alpha fragment
DAEFRHDSGYEVHHQK

SEQ ID NO: 4 - protein, 34 aa, Artificial Sequence
  human APPs alpha fragment with 2xHA tag
YPYDVPDYAYPYDVPDYADAEFRHDSGYEVHHQK

SEQ ID NO: 5 - protein, 26 aa, Homo sapiens
  ApoE4 fragment (26 aa peptide)
YLRVRLASHLRKLRKRLLRDADDLYC

SEQ ID NO: 6 - protein, 16 aa, Drosophila melanogaster
  Penetratin
RQIKIWFQNRRMKWKK

SEQ ID NO: 7 - protein, 16 aa, Human immunodeficiency virus 1
  TAT-peptide
CGRKKKRRQRRRPPQC

SEQ ID NO: 8 - protein, 27 aa, Artificial sequence
  model amphiphatic peptide MAP
GALFLGFLGAAGSTMGAWSQPKSKRKV

SEQ ID NO: 9 - protein, 9 aa, Artificial sequence
  artificially designed CPP R9
RRRRRRRRR

SEQ ID NO: 10 - protein, 18 aa, Mus musculus
  MOD_RES 18..18: AMIDATION
LLIILRRRIRKQAHAHSK

SEQ ID NO: 11 - protein, 28 aa, Homo sapiens
  MOD_RES 28..28: AMIDATION
GWTLNSAGYLLGKINLKALAALAKISIL

SEQ ID NO: 12 - protein, 27 aa, Human immunodeficiency virus
  MPG
GALFLGFLGAAGSTMGAWSQPKSKRKV